(19) United States
(12) Patent Application Publication
(10) Pub. No.: US 2019/0128845 A1
(43) Pub. Date: May 2, 2019

(54) IMMS METHOD FOR PETROLEUM FEEDSTOCK EVALUATION

(71) Applicants: Ohio State Innovation Foundation, Columbus, OH (US); Chevron U.S.A. Inc., San Ramon, CA (US)

(72) Inventors: Aamena Parulkar, Columbus, OH (US); Nickolas A. Brunelli, Columbus, OH (US); Matthew R. Hurt, Richmond, CA (US); Joshua Allen Thomson, Martinez, CA (US); Bi-Zeng Zhan, Albany, CA (US)

(21) Appl. No.: 16/179,837

(22) Filed: Nov. 2, 2018

Related U.S. Application Data
(60) Provisional application No. 62/580,977, filed on Nov. 2, 2017.
(60) Provisional application No. 62/640,088, filed on Mar. 8, 2018.

(51) Int. Cl. G01N 27/62; G01N 33/28; H01J 49/40; H01J 49/42; H01J 49/16

(52) U.S. Cl. CPC G01N 27/622; G01N 33/2835; H01J 49/165; H01J 49/42; H01J 49/40

(57) ABSTRACT

An ion mobility mass spectrometry (IMMS) method is disclosed for evaluating petroleum feedstock compositions. The method is useful to determine, e.g., nitrogen speciation in chemical components of a petroleum composition and may be used to evaluate hydroprocessing catalyst performance.

### CROSS-REFERENCE TO RELATED APPLICATIONS

This application is related to, and claims priority benefit from, U.S. Provisional Application Ser. No. 62/580,977, filed Nov. 2, 2017, entitled “IMMS METHOD FOR PETROLEUM FEEDSTOCK EVALUATION”, and related to, and claims priority benefit from, U.S. Provisional Application Ser. No. 62/640,088, filed Mar. 8, 2018, entitled “IMPROVING HYDRODENITROGENATION CATALYST PERFORMANCE THROUGH ANALYZING HYDROTREATED VACUUM GAS OIL USING ION MOBILITY-MASS SPECTROMETRY”, each of which is herein incorporated by reference in its entirety.

### FIELD OF THE INVENTION

The invention concerns an ion mobility mass spectrometry (IMMS) method for evaluating petroleum feedstock compositions. The method is useful to determine, e.g., nitrogen speciation in chemical components of a petroleum composition and may be used to evaluate hydroprocessing catalyst performance.

### BACKGROUND OF THE INVENTION

Advances in catalytic materials for refining technology are required to process heavier crude to produce valuable chemicals and fuels. Upgrading heavy crudes such as vacuum gas oil (VGO) can be accomplished using hydrotreating processes, which includes hydrodenitrogenation (HDN), hydrodesulfurization (HDS), hydrodemetallization (HDM), hydrodeoxygenation (HDO), and hydrodearomatization (HAD).^{1,2} Of these multiple processes, HDS and HDN are of major importance, as the environmental regulations require the nitrogen (N) and sulfur (S) content to be at sub-PPM levels in the final product. In deep HDN and HDS processes, catalyst design becomes very important, as the catalyst needs to be both active and resistant to corrosive species such as polyaromatic species containing pyridinic nitrogen. The complex nature of hydrocarbonaceous feeds makes it difficult to understand the structure-function relationships, impeding the discovery of new catalytic materials. The process can be improved through interdisciplinary efforts to use analytical methods for investigating effect of catalyst design on efficiency of hydrotreating processes.

The catalytic process for HDN and HDS used for upgrading VGO involves treating the crude feed at high temperature and pressure with hydrogen over catalytic materials to saturate the petroleum fractions and remove heteroatoms including N and S. Both HDN and HDS are typically performed using self-supported and supported transition metal sulfides, carbides, or nitrides specifically of molybdenum and tungsten with nickel or cobalt promoters.³⁻⁷ Efforts continue to improve the formulation and performance of these catalysts with real gains in performance for both the HDN and HDS process. However, understanding the catalytic performance for HDN is critical because the nitrogen species can severely inhibit HDS, cause catalyst deactivation in downstream processes, and make the hydroprocessed oils sensitive to light.⁸⁻¹¹ Development of better catalysts for HDN can be expedited by understanding both the feed and treated sample composition. The nitrogen species are classified in two categories: (i) aliphatic amines and anilines; (ii) and heterocyclic N species sub-classified into pyridinic (basic) and pyrrolic (non-basic) species.¹² HDN catalysts need to treat each of these species, which is challenging considering the inherent chemical differences between all of these species.

One strategy to understand the effect of these different N species on HDN catalysis is to test model compounds. While studies performed using model compounds have provided insights about the catalytic process including HDN pathways, active sites, and effect of reaction conditions, real world samples involve significant complexity that can impact catalyst performance in real feeds.^{6,13-18} Recent reports suggest use of real feeds to develop structure function relationships for catalytic systems, but it is very important to investigate the different types of species present in the feed to continue improving the design of catalysts.¹⁹

Research that characterizes the composition of petroleum-petroleomics—has used multiple techniques including gas chromatography with flame ionization detection (GC-FID), GC with atomic emission detector (GC-AED), GC coupled with mass spectrometry (GC-MS), and high-resolution mass spectrometry (HR-MS).^{20,21} Initial studies involved GC-FID and GC-MS; these techniques are capable of analyzing most species that are lower boiling point and non-polar in nature.^{22′23} Often, GC methods result in co-elution of isomeric species, which can prevent the compositional analysis of samples, limiting the insight into the catalyst performance for removal of different species. More information can be gleaned using high-resolution mass spectrometry to extract information about contaminant and additives in crude oil, composition of asphaltenes, and heteroatomic species in oil samples.^{11,23-27} While methods such as Fourier transform ion cyclotron resonance mass spectrometry (FT-ICR MS) have considerable resolution,^{11,24,28,29} sample complexity remains a challenge that can be addressed through using two dimensional analytical methods.

An important two-dimensional technique that provides information about both the molecular composition and the structure is called ion-mobility mass spectrometry (IMMS).³⁰ In IMMS, the first stage is an ion mobility cell that can separate gas phase ions based on their collision cross section (CCS), which is related to the structure and allows differentiation of isomers.³¹ Indeed, recent work was able to assign structures for 6 different polyaromatic species with same elemental composition (C₂₃H₂₆) utilizing the ion-mobility data.₃₂ Additional reports have demonstrated the benefit of using IMMS for de-convoluting the complexities of crude oil samples.^{23,33-37} Santos and coworkers demonstrated the benefit of IMMS for characterization of complex petroleum samples by resolving peaks corresponding to contaminants and additives.²³

Analysis of complex mixtures using mass-spectrometric techniques has a strong dependence on the sample preparation and type of ion sources used. In a study by Scharder, it was demonstrated that different species are observed as the ionization technique is varied.₃₈ Another factor that plays a role is the solvent, as differences in the solubilities of different species can influence the ionization efficiency.^{39,40} Most common solvent systems used for petroleomics include the use of toluene or a mixture of methanol and toluene to prepare the dilute solutions of oil samples.^{28,41,42} One other solvent that has been reported for studying petroleum based samples is dichloromethane (DCM).^{25,43} One specific property of DCM can be seen from the studies done by Gray and Jokuty for investigating nitrogen species in gas oils, which report that DCM is more efficient in extracting pyrrolic nitrogen species as compared to methanol.^{8,44} Therefore, solvent selection is important to ensure that both the classes of nitrogen, pyridinic and pyrrolic, are being transferred efficiently to gas phase ions. Another strategy to enhance the ionization efficiency is to use an additive like an organic acid (e.g., formic acid, acetic acid) or base (e.g., ammonium hydroxide) that can facilitate the ionization.⁴⁵ Different combinations of solvent and additives can allow us to access and extract a range of information from the samples.

Despite the advances in analytical techniques, including those made in ion mobility mass spectrometry, and in the understanding of hydroprocessing catalyst performance, a continuing need exists for solutions to the problem of providing improvements in such techniques and methods, and in the understanding and design of hydroprocessing catalysts.

Additional background information related to this invention is provided in the publications and patents identified in the publications section of this application. Where permitted, each of these publications is incorporated herein by reference in its entirety.

### SUMMARY OF THE INVENTION

The present invention is directed to an ion mobility mass spectrometry (IMMS) method for determining the nitrogen compound speciation in a petroleum composition. The method generally includes providing a sample of a petroleum composition that is combined with a solvent and an ionization enhancer to form an IMMS sample suitable for use with the IMMS system. The IMMS sample is typically ionized in an ionization source, such as an electrospray source. Ions are then passed to the ion mobility mass spectrometer, and mass and drift time spectra of ionized IMMS sample components are obtained.

The invention is also directed to the use of the IMMS system and method to evaluate the catalytic performance of hydroprocessing catalysts, including the effectiveness of such catalysts to remove nitrogen from a petroleum composition. The method generally includes providing at least two petroleum samples from a petroleum composition with one or more of the samples being unprocessed, i.e., not hydroprocessed by contacting with a hydroprocessing catalyst, and one or more of the samples being hydroprocessed by contacting the sample(s) with the hydroprocessing catalyst under hydroprocessing conditions. Each set of samples is then separately used to form IMMS samples that are then separately provided to the IMMS system to obtain mass and drift time spectra of ionized IMMS sample components for each of the two unhydroprocessed and hydroprocessed IMMS sample sets. The spectra results are then analyzed to assign chemical species to selected peaks of the mass spectra and to determine the double bond equivalent (DBE) of the first and second IMMS samples relative to carbon number.

### BRIEF DESCRIPTION OF THE DRAWINGS

The scope of the invention is not limited by these representative figures and is to be understood to be defined by the claims of the application.

FIG. 1 shows mass spectra of carbazole used as model compound to test different solvent systems for IMMS analysis:dichloromethane (DCM) with 0.05% trifluoroacetic acid (TFAA) (top) and 1:1 v/v methanol/toluene with 0.04% formic acid (bottom).

FIG. 2 shows a comparison of mass spectra of VGO feed solution prepared in different solvent systems for IMMS analysis:dichloromethane (DCM) with 0.05% trifluoroacetic acid (TFAA) (top) and 1:1 v/v methanol/toluene with 0.04% formic acid (bottom).

FIG. 3 shows mobilograms of the hydrotreated samples treated with catalyst A (N: 2.4 PPM), C (N: 1.6 PPM), and layered bed C-A (N: 0.9 PPM) respectively for deep HDN. The species below the highlighted region indicate the most compact species, which are found to be oxygenated species from PetroOrg analysis.

FIG. 4 shows DBE vs. Carbon# plots for the hydrotreated samples treated with catalyst A (N: 2.5 PPM), C (N: 1.6 PPM), and layered bed CA (N: 0.9 PPM) respectively for deep HDN. Feed treated on the layered catalyst bed has the lowest N concentration indicating that the synergy between the two catalysts.

FIG. 5 shows mass spectra of the hydrotreated samples treated with catalyst A (N: 59 PPM), B (N: 50 PPM), and C (N: 19 PPM) for moderate nitrogen conversion. The comparisons show that the behavior of the three catalysts is different, with catalyst C being the most efficient in removing high molecular weight species from the feed.

FIG. 6 shows DBE vs. Carbon# plots for the hydrotreated samples treated with catalyst A (N: 59 PPM), B (N: 50 PPM), and C (N: 19 PPM) respectively for moderate nitrogen conversion. The treated sample have similar bulk distribution of N1 class species however samples treated with A and B also contain low DBE-low C# species attributed to carbazole and benzocarbazole type species.

FIG. 7 shows illustrative mobilograms of the hydrotreated samples treated with catalyst A (N: 59 PPM), B (N: 50 PPM), and C (N: 19 PPM) respectively for moderate HDN. The highlighted region indicates the differences in the most compact species, which are found to be oxygenated species from PetroOrg analysis.

### DETAILED DESCRIPTION

Detailed description and information related to this invention is provided in the publications and patents identified in the publications section of this provisional application. Where permitted, each of these publications is incorporated herein by reference in its entirety. The claims provided in this application further describe the scope of the invention, as well as specific embodiments within the scope of the invention. Where any dependent claim refers to one or more previous claims, it is to be understood that all such combinations of claimed features are within the scope of the invention, regardless of whether or not a specific combination of features is explicitly stated.

The present invention makes use of an ion mobility mass spectrometry (IMMS) method to aid in determining the nitrogen compound speciation in a petroleum composition. The IMMS method is straightforward and comprises providing a sample of a petroleum composition; combining the petroleum sample with a solvent and an ionization enhancer to form an IMMS sample; providing the IMMS sample to an ion mobility mass spectrometer; and obtaining the mass and drift time spectra of ionized IMMS sample components.

Various petroleum feedstocks may be used with the method, including, e.g., petroleum compositions selected from vacuum gas oil, vacuum resid, aromatic resid, unconverted crude oil, coker gas oil, cycle oil, straight run diesel, or a mixture thereof. In one embodiment, the petroleum composition comprises a vacuum gas oil, or consists essentially of a vacuum gas oil, or is a vacuum gas oil.

Although a variety of solvents may be used, particularly suitable solvents may be selected from acetonitrile, dichloromethane, dichloroethane, tetrahydrofuran, methanol, ethanol, propanol, nitromethane, toluene, water, dimethylformamide, dimethylsulphoxide, or a mixture thereof. In particular embodiments, the solvent comprises dichloromethane, or consists essentially of dichloromethane, or is dichloromethane.

The method also makes use of an ionization enhancer. While various compounds may be known as being useful ionization enhancers in the art, the present invention ionization enhancer is selected from an organic acid, a halogenated organic acid, a carboxylic acid, a halogenated carboxylic acid, or a mixture thereof. In particular embodiments, the ionization enhancer comprises a halogenated organic acid, or consists essentially of a halogenated organic acid, or is a halogenated organic acid. In more particular embodiments, the ionization enhancer is a halogenated carboxylic acid, or a halogenated acetic acid, or a fluorinated acetic acid, or a chlorinated acetic acid, or fluoroacetic acid, or chloroacetic acid, or difluoroacetic acid, or dichloroacetic acid, or trifluoroacetic acid (TFAA), or trichloroacetic acid, or a mixture thereof. In still further particular embodiments, the ionization enhancer comprises trifluoroacetic acid, or consists essentially of trifluoroacetic acid, or is trifluoroacetic acid.

Although the amount of the ionization enhancer is not generally limited, typical ranges are from greater than 0% v/v to 0.2% v/v, more particularly from about 0.01 to about 0.15% v/v, or from 0.02 to about 0.10% v/v, 0.02 to about 0.0.08% v/v, or 0.02 to about 0.06% v/v. As described in the examples, the ionization enhancer may be used in an amount of about 0.05% v/v, in particular an amount of about 0.05% v/v of trifluoroacetic acid

The ionization enhancer may also be specified according to a pKa value. For example, in an embodiment, the ionization enhancer has a pKa value that is substantially lower than acetic acid at the same temperature and in the same solvent. More particularly, in related embodiments, the ionization enhancer may have a pKa value that is less than the pKa value of acetic acid at the same temperature and in the same solvent by at least 80%, or 70%, or 60%, or 50%, or 40%, or 30%, or 20%, or 10%. The ionization enhancer may also be specified as having a pKa value in the range from about 0.0 to about 4.5 at 25° C. in water.

The mass spectrometer itself is a conventionally-known analytical instrument that comprises or uses an electrospray source as an ionization source. In general, the ion mobility mass spectrometer comprises an ionization source, a travelling wave ion guide, a quadrupole, a tri-wave ion mobility separator, and a time-of-flight mass analyzer. In operation, the IMMS sample is provided to the electrospray source, wherein the IMMS sample is ionized by the electrospray source and ions from the IMMS sample are thereby provided to the mass spectrometer.

The invention is further directed to a method for determining the effectiveness of a hydroprocessing catalyst in removing nitrogen-containing compounds from a petroleum composition. The method comprises providing first and second petroleum samples, with the first petroleum sample being from a petroleum composition that has not been hydroprocessed using a hydroprocessing catalyst and the second petroleum sample being from the same petroleum composition that has been hydroprocessed by contacting the second petroleum sample with the hydroprocessing catalyst under effective hydroprocessing conditions. The first and second samples are separately combined with portions of the same solvent and the same ionization enhancer to form corresponding first and second IMMS samples. Each of the first and second IMMS samples is then provided to an ion mobility mass spectrometer, with mass and drift time spectra of ionized IMMS sample components obtained for each of the first and second IMMS samples. The spectra may then be analyzed to assign chemical species to selected peaks of the mass spectra and to determine the double bond equivalent (DBE) of the first and second IMMS samples relative to carbon number.

Each of the foregoing descriptions for the petroleum composition, solvents, and the ionization enhancer also apply for the method for determining the effectiveness of a hydroprocessing catalyst in removing nitrogen-containing compounds.

The following examples and discussion illustrate and describe non-limiting aspects of the invention.

### Examples

### Materials

High purity (HPLC grade) methanol (MeOH), toluene, and dichloromethane (DCM) from Fisher Chemicals, trifluoroacetic acid (TFAA 99%, Acros), and formic acid (FA, Amresco) are used for sample preparation. Chevron Corporation provided seven samples, including the feed and the hydrotreated samples for this study. All chemicals and VGO samples are used as received without any further purification.

### Synthesis and Characterization of Catalytic Materials

Three catalysts are prepared using previously reported methods with the brief synthesis procedure mentioned in the following sections. The catalysts had compositions of NiₓW_{y} on an alumina/zeolite support, NiₓMo_{y}P_{z} on an alumina support, and a CoₓMo_{y}W_{z} on a methocel support. The catalyst characterization included X-ray diffraction and BET analysis. Catalyst characterization details can be found in the respective cited patents.

Preparation of Supported Nickel Tungsten (NiₓW_{y}—Catalyst A)

The base for making catalyst A was prepared according to method described in U.S. Pat. No. 9,187,702 B2. Silica-alumina powder (obtained from Sasol) of 67 g (dry weight, weighed after drying the sample at 593° C.), pseudo boehmite alumina powder (obtained from Sasol) of 25 g (dry weight) and 8 g of zeolite Y (from Tosoh) were mixed well. A 1M HNO₃ acid aqueous solution (1 wt. % of dry catalyst base) was added to the mix powder to form an extrudable paste. The paste was extruded in 1/16″ asymmetric quadrilobe shape and dried at 120° C. overnight. The dried extrudates were calcined at 593° C. for 1 h with purging excess dry air and cooled down to room temperature.

Impregnation of Ni and W was done using a solution containing ammonium metatungstate and nickel nitrate to the target metal loadings of 4 wt. % NiO and 28 wt. % WO₃ in the finished catalyst. 3-carboxy-3-hydroxy-pentanedioic acid at the amount of 10 wt. % of finished dry catalyst was added to the Ni/W solution. The solution was heated to above 50° C. to ensure a completed dissolved (clear) solution. The total volume of the metal solution matches the 103% water pore volume of the base extrudates (incipient wetness method). The metal solution was added to the base extrudates gradually while tumbling the extrudates. When the solution addition was completed, the soaked extrudates are aged for 2 h. Then the extrudates are dried at 120° C. overnight. The dried extrudates are calcined at 205° C. for 2 h with purging excess dry air and cooled down to room temperature.

Preparation of Unsupported Nickel Molybdenum Tungstate (NiₓMo_{y}W_{z}—Catalyst B)

Catalyst B was prepared according to method described in U.S. Pat. No. 8,173,570 B2: 52.96 g of ammonium heptamolybdate (NH₄)₆Mo₇O₂₄.4H₂O was dissolved in 2.4 L of deionized water at room temperature. The pH of the resulting solution was within the range of 5-6. 73.98 g of ammonium metatungstate powder was then added to the above solution and stirred at room temperature until completely dissolved. 90 mL of concentrated (NH₄)OH was added to the solution with constant stirring. The resulting molybdate/tungstate solution was stirred for 10 min and the pH monitored. The solution has a pH in the range of 9-10. A second solution was prepared containing 174.65 g of Ni(NO₃)₂.6H₂O dissolved in 150 mL of deionized water and heated to 90° C. The hot nickel solution was then slowly added over 1 h to the molybdate/tungstate solution. The resulting mixture was heated to 91° C. and stirring continued for 30 min. The pH of the solution was in the range of 5-6. A blue-green precipitate forms and the precipitate was collected by filtration. The precipitate was dispersed into a solution of 10.54 g of maleic acid dissolved in 1.8 L of DI water and heated to 70° C. The resulting slurry was stirred for 30 min at 70° C., filtered, and the collected precipitate vacuum dried at room temperature overnight. The material was then further dried at 120° C. for 12 h. The prepared powder of catalyst B has a formula of (NH₄) {[Ni_{2.6}(OH)_{2.08}(C₄H₂O₄₂₋)_{0.06}] (Mo_{0.35}W_{0.65}O₄)₂}. The resulting material has a typical XRD pattern with a broad peak at 2.5 Å, denoting an amorphous Ni—OH containing material. The BET Surface area of the resulting material was 101 m₂/g, the average pore volume was around 0.12-0.14 cm₃/g, and the average pore size was around 5 nm. 40 g of catalyst B powder prepared was mixed with 0.8 g of methocel, (a commercially available methylcellulose and hydroxypropyl methylcellulose polymer from Dow Chemical Company), and approximately 7 g of DI water was added. Another 7 g of water was slowly added until the mixture was of an extrudable consistency. The mixture was then extruded in 1/12″ asymmetric quadrilobe shape and dried under N₂ at 120° C. prior to catalysis testing.

Preparation of Supported Nickel Molybdenum Phosphide (NiₓMo_{y}P_{z}—Catalyst C)

Catalyst C was prepared according to US20140367311 A1. An alumina containing slurry was prepared as follows: to a tank was added 13630 L of city water. The temperature was brought to 49° C. with heating. An aluminum sulfate stream and a sodium aluminate stream are added continuously to the tank under agitation. The aluminum sulfate stream consists of an aqueous solution of aluminum sulfate (containing 8.3 wt. % Al₂O₃, 20 gal/min) inline diluted with water (79.9 L/min), while the sodium aluminate stream was composed of an aqueous solution of sodium aluminate (containing 25.5 wt. % Al₂O₃) inline diluted with water (35.3 gal/134 L/min). The addition speed of the sodium aluminate solution in the sodium aluminate stream was controlled by the pH of the alumina slurry. The pH was controlled at 9.0 and temperature at 49° C. The temperature control was achieved through adjusting the temperature of dilution water for both streams. After 2,082 L of the aqueous solution of sodium aluminate was added to the tank, both aluminum sulfate and sodium aluminate streams are stopped. The temperature of the resulting slurry was increased to 53° C. with steam injection for 35 min. Both aluminum sulfate and sodium aluminate streams are resumed while the steam injection was kept on. During this step, the pH of the slurry was kept at 9.0, while the temperature was allowed to rise freely. The precipitation was stopped once 4542 L of the aqueous aluminum sulfate solution was added. The final temperature of the slurry reaches 65° C. After the precipitation was stopped, the pH was raised with addition of the same aqueous sodium aluminate to 9.3. The alumina slurry was then filtered and washed to remove Na₊ and SO₄₂₋. This slurry is referred to as slurry A.

After about half of slurry A was pumped to another tank, it was heated to 60-66° C. with steam injection and maintained at this temperature. MS-25 (63.5 kg) was added to the tank. The amount of MS-25 was controlled so that the final support contained 3% SiO₂. Acetic acid (113 kg, 29.2%) was subsequently added to the slurry before it was agitated for 30 min. After the agitation, ammonia (60.8 kg, 6.06%) was added before the slurry was filtered to give a cake. The obtained cake was dried at about 288° C. to give an alumina powder containing about 60% moisture. The powder was next transferred to a mixer and treated with 0.5% HNO₃ and 10% of recycle catalyst/support fines. The mixture was kept mixing until an extrudable mixture was formed. The mixture was then extruded in 1/16″ asymmetric quadrilobe shape, dried, and calcined at 732° C. to give a catalyst support.

The support was impregnated with an aqueous Ni—Mo—P metal solution to give a catalyst containing 25.6% molybdenum oxide, 5.0% nickel oxide, and 4.5% phosphorus oxide. The catalyst C showed surface area and pore volume of 152 m₂/g and 0.41 mL/g by N₂ adsorption.

### Hydrotreating of Vacuum Gas Oil Samples

The VGO feedstock used for this study was a straight run VGO directly from the crude distillation with properties listed in Table S1.

TABLE S1
Properties of VGO Feed

| | |
|---|---|
| Density, g/mL | 0.924 |
| Nitrogen content, PPM | 997 |
| Sulfur content, wt. % | 2.21 |
| Hydrogen content, wt % | 12.27 |

Components by MS, Vol %

| | |
|---|---|
| Parafins | 14.9 |
| Naphthenes | 29.0 |
| Aromatics | 35.3 |
| Sulfur compounds | 20.0 |

Simulated Distillation, ° C. @ wt %

| | |
|---|---|
| IBP | 330 |
| 5% | 365 |
| 10% | 384 |
| 15% | 384 |
| 20% | 405 |
| 30% | 422 |
| 40% | 437 |
| 50% | 450 |
| 60% | 465 |
| 70% | 480 |
| 80% | 499 |
| 90% | 523 |
| 95% | 543 |
| EP | 587 |

The hydrotreating method was conducted using an in-house designed fixed-bed hydroprocessing unit equipped with an automated catalyst and distillation system. Catalyst extrudates (L/D=1-2) of 6 mL are loaded to a stainless-steel reactor. The catalyst bed was packed with 100-mesh alundum to improve feed-catalyst contact and to prevent channeling and was placed in the isothermal zone of furnace.

Catalysts were sulfided in-situ before contact with the VGO feedstock. Adsorbed moisture was removed by drying the catalyst at 120° C. for 2 h under N₂ flow. Flow was then switched to hydrogen and unit pressure was increased to 55 bar. Hydrogen flow was controlled at 134 mL/min. The catalyst was then exposed to a stream at 9 mL/h, which was a diesel containing 2.5% DMDS. The process conditions are maintained for a total of 10 h from the time the sulfiding feed was started. This was to ensure the catalyst was fully wetted by the sulfiding feed. The reactor temperature was raised to 345° C. at 0.5° C./min and held for 5 h. The unit pressure was increased to 159 bar when sulfiding was completed. The flow was then switched to VGO feedstock and reactor temperature was raised to 371° C.

Hydrotreating of VGO feedstock was performed at linear hourly space velocity (LHSV) of 2,236 mL/min of once-through hydrogen flow, and 159 bar of hydrogen inlet pressure. The liquid product was sent to an on-line distillation for a cut point controlled at 316° C. Samples from the distillation overhead (DO), distillation bottom (DB), and off gas are collected and analyzed daily for S and N content in DB and for hydrocracking conversion calculation. Reactor temperature was controlled at 644, 655 and 666 K, respectively for all the three catalysts to generate DB products with different N content for MS study. The VGO starting material was hydrotreated over different HDN catalysts to get either moderate N conversion or high N conversion. To differentiate samples, the notation used was X-#PPM, where X corresponds to the catalyst (A-C) and #PPM is the concentration of N in the treated sample. The nitrogen content was determined using X-ray fluorescence spectroscopy and the values are shown in Table 1.

TABLE 1
Properties of feed and hydrotreated
samples using different catalysts

| | Total N | Mean | HDN |
|---|---|---|---|
| | Content | Molecular | Temperature |
| Sample ID | (PPM) ^{a} | Weight ^{b} | (° C.) ^{c} |
| Feed | 997 | | — |
| Moderate HDN | | | |
| Catalyst A | 59 | 437.12 | 382 |
| Catalyst B | 50 | 454.43 | 371 |
| Catalyst C | 19 | 409.83 | 382 |
| Deep HDN | | | |
| Catalyst A | 2.4 | 351.95 | 393 |
| Catalyst C | 1.6 | 389.15 | 393 |
| Layered Bed: Catalyst A-C | 0.9 | 349.69 | 393 |

^{a} Determined using X-ray fluorescence.
^{b} Determined from analyzing the spectrum using the PetroOrg software.
^{c} Reaction temperature used for hydrotreating the feed.

Sample Analysis with Ion Mobility Mass Spectrometry

Model compounds and the VGO samples solutions were prepared at a concentration of 1 mg/mL by dissolving an appropriate amount of sample in dichloromethane. Trifluoroacetic acid (TFAA) was added (0.05% (v/v)) to enhance the ionization of the sample. The ESI-TWIM-MS (electrospray travelling wave ion mobility mass spectrometry) experiments are performed on a Waters Synapt G2-S high definition mass spectrometer. The instrument is a hybrid quadrupole ion-mobility orthogonal acceleration time-of-flight (TOF) mass spectrometer. The instrument parameters are optimized to achieve stable electrospray. Instrument parameters used for collecting IMMS data are shown in Table S2.

TABLE S2
ESI-IMMS instrument parameters

| Parameter | Optimized value |
|---|---|
| Capillary voltage (kV) | 2.5 |
| Sample cone voltage (V) | 20 |
| Source temperature (° C.) | 150 |
| Desolvation temperature (° C.) | 200 |
| Desolvation gas (N2) flow rate (L/h) | 500 |
| m/z range | 50-2000 |
| LM/HM resolution | 15/20 |
| Wave velocity (m/s) | 650 |
| Wave height (V) | 40 |
| Drift gas pressure (mbar) | 2.75 |

### Results

### Model Pyrrolic Compounds

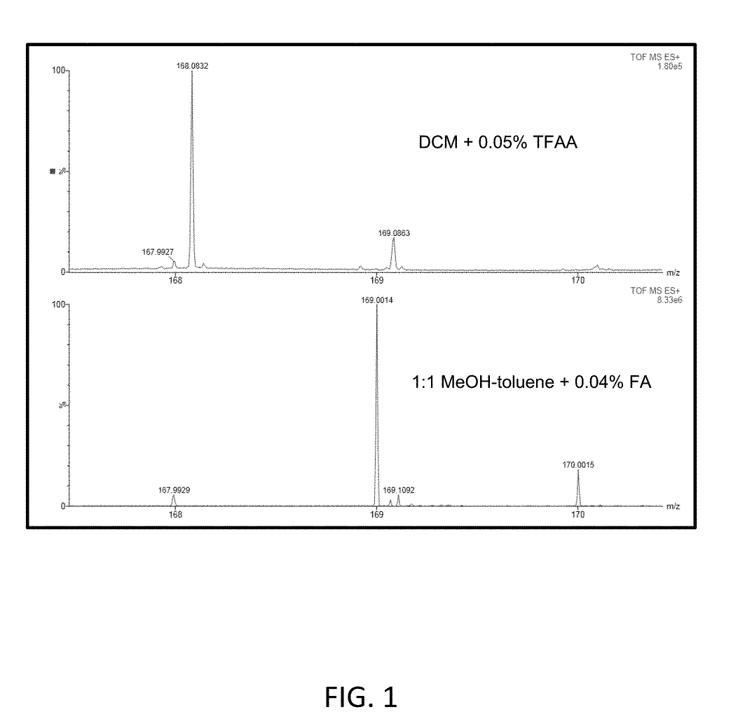

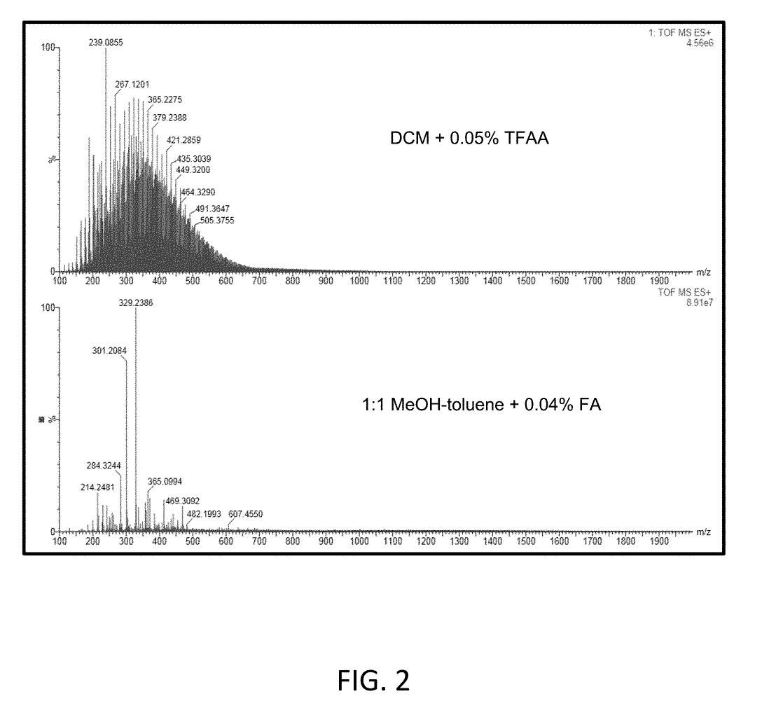

Initial work focused on investigating the solvent system for analyzing the VGO samples. For this, the ability to detect carbazole as a molecular ion using the ESI-IMMS system was investigated. The 1 mg/mL solution of carbazole was prepared in two solvent systems, 1:1 v/v methanol/toluene with 0.04% v/v formic acid used previously and DCM with 0.05% v/v TFAA.⁴¹⁻⁴² The molecular ion for carbazole was observed only for the sample prepared in DCM with 0.05% TFAA as shown in FIG. 1. Testing these two solvent systems for the feed sample reveals that more species are observed when using 0.05% v/v TFAA/DCM as compared to methanol/toluene with 0.04% formic acid, as shown in FIG. 2. This suggests that ionization occurs more efficiently for the DCM solvent system than the methanol/toluene mixture. Therefore, for the rest of this work all samples were prepared in DCM with 0.05% v/v TFAA. It should also be noted that some of the peaks in the sample MS correspond to the species present in the solvent system. These peaks appear despite using HPLC grade DCM. Therefore, analysis was performed by comparing the sample MS with the blank solvent MS.

### VGO Feed Analysis

The catalyst performance was determined through feeding a VGO material containing 997 PPM nitrogen to the hydrotreating reactor. Initial tests targeted different catalysts for moderate to high conversion of N in the feed. To understand the effect of catalyst on the type of species removed the IMMS data for the treated samples was compared to the feed.

The mass spectrum of the feed shows a complex mixture with broad distribution consisting of multiple overlapping Gaussian distributions. However, the overall distribution of the species seems centered on ^{˜}350 m/z. The MS also has a low intensity tail showing presence of high molecular weight species>600 m/z in low concentration. One possible reason for the low intensity of peaks in high m/z range (>600 m/z) might be the suppression of ionization of heavy species in presence of more polar low m/z species.

Three different sets of samples were analyzed to assess the performance of different catalysts for HDN of this particular feed: (1) deep HDN, where treated samples contain <2.5 PPM N, (2) moderate HDN, where treated samples contain between 15-60 PPM N, and (3) layered bed catalyst for deep HDN, where catalyst A and catalyst C are used in combination.

Analysis of Samples Hydrotreated with Single Catalyst

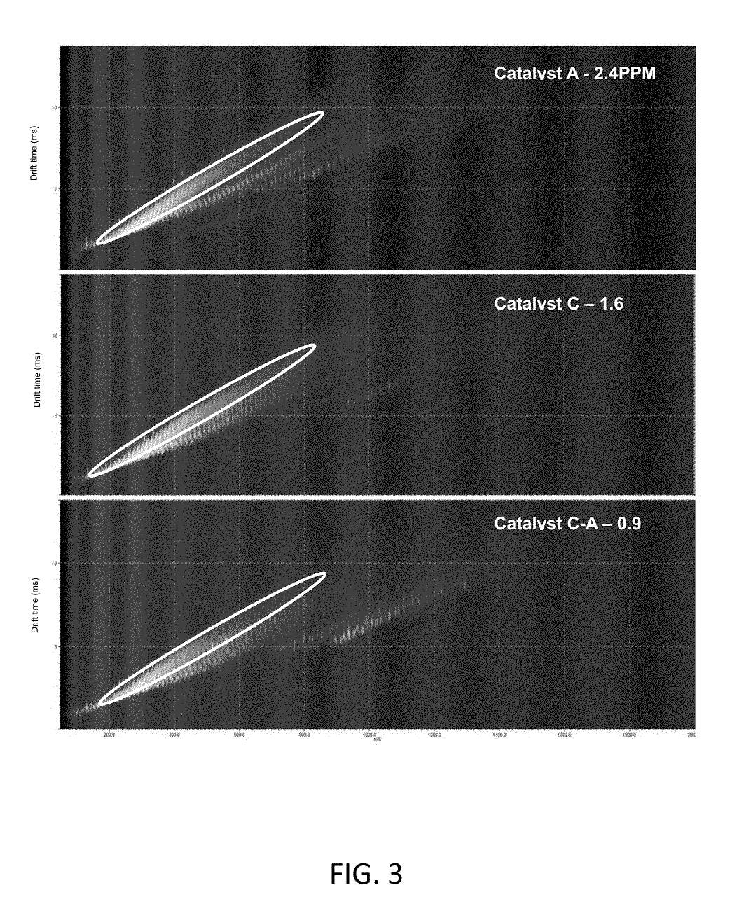

In the first set, catalyst A and C are tested for deep HDN to treat the feed and reduce the nitrogen concentration below 2.5 PPM. For these experiments, the total volume, space velocity, and temperature are held constant. The samples are analyzed using IMMS to produce a mobilogram. A mobilogram is a heat map of drift time vs. m/z with the slope representing compactness of species. The mobilograms for these samples have similar trends with the main difference in the abundance of the most compact species, as highlighted in FIG. 3. Different sections of the mobilogram can be extracted and analyzed separately, which simplifies the peak assignment process through PetroOrg.⁴⁶ The most compact species lying on the smallest slope on the mobilogram are extracted and analyzed separately to identify the differences. The peaks in the extracted region are associated with oxygenated species formed by atmospheric oxidation during the shelf life of the sample. These types of species are not expected to form during the refining process but occur because of sample sensitivity to oxygen and light that are unavoidable. The data excluding the oxygenated species was analyzed separately.

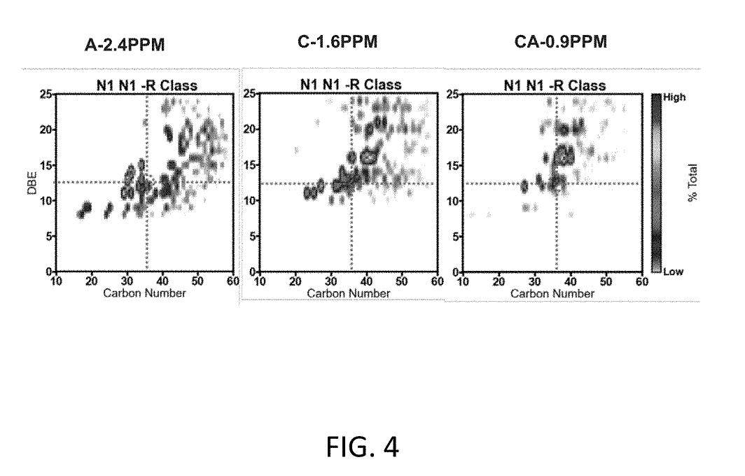

The species assigned to N1 class are then plotted on a double bond equivalent (DBE) vs. carbon number plot to understand the selectivity of catalysts for different types of species including aliphatic vs. aromatic species. The software is powerful and uses the molecular weight known to high precision to select compounds that contain single nitrogen. Even though the distribution of species at these N levels look similar in MS, slight differences in the type of species removed can be observed on DBE vs. carbon number plot for N1 class, as shown in FIG. 4. Catalyst C more effectively removes species with low carbon# and low DBE in addition to reducing the N concentration to a lower level indicating higher activity for bulk HDN. This indicates that the catalyst design can influence both the activity and selectivity of HDN process.

For better understanding of the nitrogen removal process, the next set of samples analyzed was for hydrotreated VGO samples that are treated to moderate N concentration of 105 of ppm. The complexity of the feed and limited time to produce samples makes it difficult to achieve exactly the same nitrogen concentration. To achieve similar nitrogen concentration after treatment, the temperature of the hydrotreater was varied over a moderate range, while maintaining the total volume and space velocity. Changing the temperature was not expected to change the mechanism or type of species processed, but rather was thought to only affect the amount of nitrogen species removed. For this work, the focus was on elucidating types of species that undergo HDN.

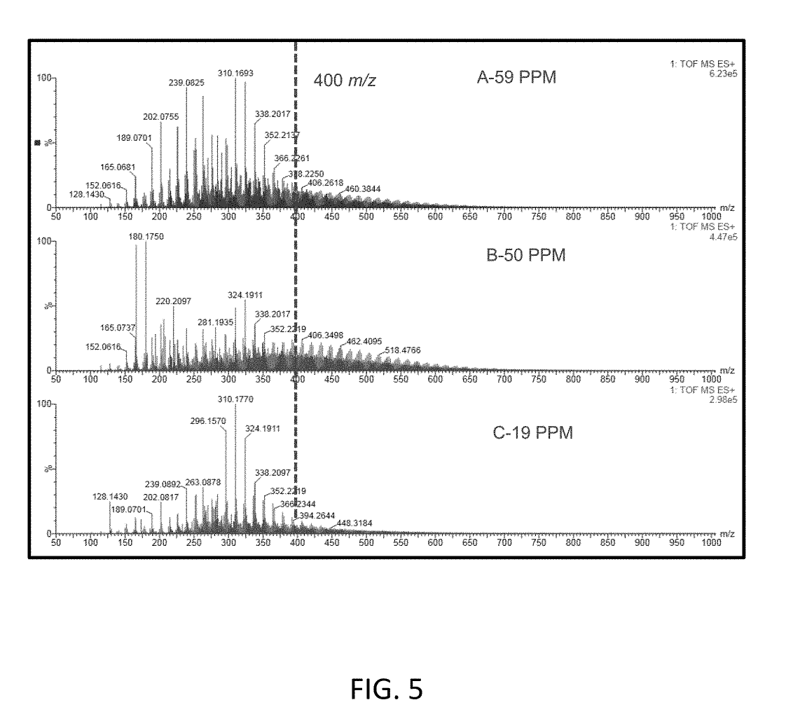

Analyzing these hydrotreated samples with IMMS revealed several interesting differences in the overall distribution of N containing species in the samples, as shown in FIG. 5. Sample B-50 PPM has the broadest distribution centered around 400 m/z whereas samples A-59 PPM and C-19 PPM are shifted towards lower m/z range. The shift in the MS is reflective of the higher efficiency of catalyst A and C to remove high molecular weight species from the feed. This property is advantageous for HDN catalyst because inhibiting effect of N-species increases with molecular heaviness.^{47,48}

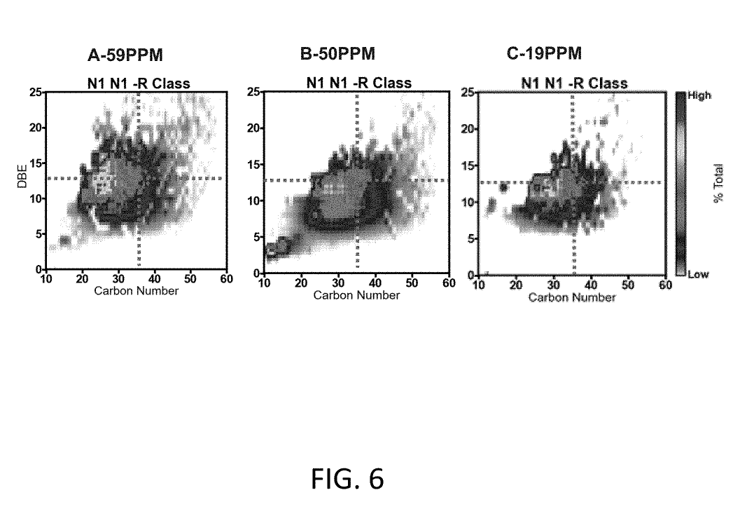

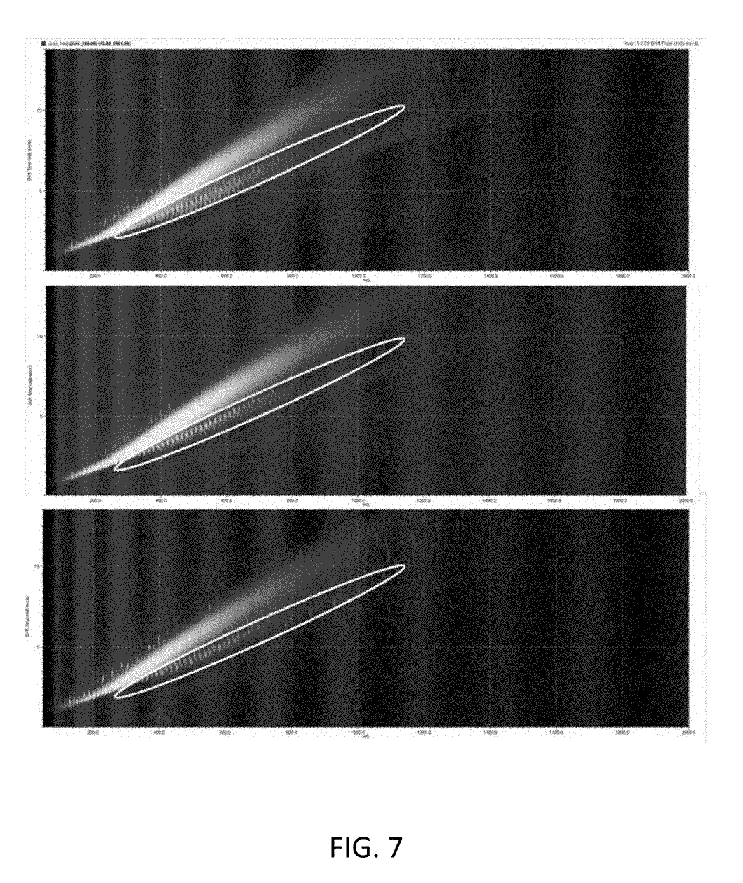

Data was extracted from the mobilograms (FIG. 7) in the same way as explained for the deep HDN samples to separate compact oxygenated species. The nitrogen speciation of all three samples was compared using the DBE vs. carbon# plot for N1 class as shown in FIG. 6, it was observed that the samples have a similar spread of species representing that the bulk sample has similar nitrogen speciation. However, the behavior of the catalysts differs slightly for the low DBE and low carbon number region, corresponding to carbazole and benzocarbazole type species. A-59 PPM and B-50 PPM show presence of species in this low DBE-low carbon number region, whereas catalyst C was observed to be more efficient in removing these species. This observation is consistent with the behavior of catalyst C for the deep HDN experiment.

Analysis of Samples Hydrotreated with Layered Catalyst Bed

Based on the above two sets of experiments, catalyst A and catalyst C are observed to have complementary behavior for HDN. From Table 1, it can be seen that using the same temperature for moderate N conversion catalyst C reduces the N concentration to 19 PPM under the same conditions, indicating higher activity of catalyst C for bulk HDN. Whereas, for deep HDN under similar conditions samples treated using catalyst A (A-2.4 PPM) and catalyst C (C-1.6 PPM) have similar N concentration. This can be attributed to the higher activity of catalyst A in the deep HDN regime. Therefore, using these two catalysts sequentially can allow more effective HDN of the feed for a given volume and space velocity. Given the higher cost of catalyst A as compared to catalyst C, this strategy improves the overall efficiency of the process while reducing the catalyst cost and preventing catalyst deactivation.

To study this, an experiment was done over a layered bed of catalyst with catalyst C upstream followed by catalyst A (20% by vol) downstream. It was observed that the combination of the two catalysts has a synergistic effect on the HDN. This results in reduction of the total N to 0.9 PPM for the layered bed system as compared to single catalysts systems. The differences in the selectivity of single vs. layered bed is more pronounced on the DBE plots. Comparing the DBE plots as shown in FIG. 3 suggests that the catalyst A and C behave slightly differently in the HDN process. Catalyst A seems more efficient in removing high DBE high carbon# species whereas catalyst C removes low DBE low carbon# species more effectively. The selectivity of the layered bed resembles more to catalyst C as it comprises 80% of the catalyst bed as well as is responsible for the bulk HDN. However, at these low N concentrations, no significant differences were observed in the N speciation in the treated sample CA-0.9 PPM as compared to A-2.4 PPM and C-1.6 PPM from the mass spectra and mobilograms. This work demonstrates a layered catalyst system is highly effective for deep HDN processing.

### PUBLICATIONS

The following footnote, cited and patent publications and provide additional information for the invention and, where permitted, are incorporated herein by reference.
(1) Bianchini, C.; Meli, A.; Vizza, F. Modelling the Hydrodenitrogenation of Aromatic N-Heterocycles in the Homogeneous Phase. Eur. J. Inorg. Chem. 2001, No. 1, 43.
(2) Jiménez, F.; Kafarov, V.; Nuñez, M. Modeling of Industrial Reactor for Hydrotreating of Vacuum Gas Oils. Simultaneous Hydrodesulfurization, Hydrodenitrogenation and Hydrodearomatization Reactions. Chem. Eng. J. 2007, 134 (1-3), 200.
(3) Katzer, J. R.; Sivasubramanian, R. Process and Catalyst Needs for Hydrodenitrogenation. Catal. Rev. 1979, 20 (2), 155.
(4) Ho, T. C.; Jacobson, A. J.; Chianelli, R. R.; Lund, C. R. F. Hydrodenitrogenation-Selective Catalysts I. Fe Promoted Mo/W Sulfides. J. Catal. 1992, 138 (1), 351.
(5) Furimsky, E. Metal Carbides and Nitrides as Potential Catalysts for Hydroprocessing. Appl. Catal. A Gen. 2003, 240 (1-2), 1.
(6) Szymańska, A.; Lewandowski, M.; Sayag, C.; Djéga-Mariadassou, G. Kinetic Study of the Hydrodenitrogenation of Carbazole over Bulk Molybdenum Carbide. J. Catal. 2003, 218 (1), 24.
(7) Lewandowski, M.; Szymańska-Kolasa, A.; Da Costa, P.; Sayag, C. Catalytic Performances of Platinum Doped Molybdenum Carbide for Simultaneous Hydrodenitrogenation and Hydrodesulfurization. Catal. Today 2007, 119 (1-4), 31.
(8) Jokuty, P. L.; Gray, M. R. Nitrogen Bases Resistant to Hydrodenitrogenation: Evidence against Using Quinoline as a Model Compound. Ind. Eng. Chem. Res. 1992, 31 (6), 1445.
(9) Oyama, S. T.; Lee, Y. K. Mechanism of Hydrodenitrogenation on Phosphides and Sulfides. J. Phys. Chem. B 2005, 109 (6), 2109.
(10) Jian, M.; Prins, R. Existence of Different Catalytic Sites in HDN Catalysts. Catal. Today 1996, 30, 127.
(11) Klein, G. C.; Rodgers, R. P.; Marshall, A. G. Identification of Hydrotreatment-Resistant Heteroatomic Species in a Crude Oil Distillation Cut by Electrospray Ionization FT-ICR Mass Spectrometry. Fuel 2006, 85 (14-15), 2071.
(12) Ho, T. C. Hydrodenitrogenation Catalysis. Catal. Rev. 1988, 30 (1), 117.
(13) Prins, R.; Egorova, M.; Röthlisberger, A.; Zhao, Y.; Sivasankar, N.; Kukula, P. Mechanisms of Hydrodesulfurization and Hydrodenitrogenation. Catal. Today 2006, 111 (1-2), 84.
(14) Bej, S. K.; Dalai, A. K.; Adjaye, J. Comparison of Hydrodenitrogenation of Basic and Nonbasic Nitrogen Compounds Present in Oil Sands Derived Heavy Gas Oil. Energy and Fuels 2001, 15 (2), 377.
(15) Ferdous, D.; Dalai, A. K.; Adjaye, J. Comparison of Hydrodenitrogenation of Model Basic and Nonbasic Nitrogen Species in a Trickle Bed Reactor Using Commercial NiMo/Al 2 O 3 Catalyst. Energy & fuels 2003, 17 (1), 164.
(16) Jian, M.; Prins, R. Mechanism of the Hydrodenitrogenation of Quinoline over NiMo(P)/Al2O3 Catalysts. J. Catal. 1998, 179, 18.
(17) Lu, M.; Wang, A.; Li, X.; Duan, X.; Teng, Y.; Wang, Y.; Song, C.; Hu, Y. Hydrodenitrogenation of Quinoline Catalyzed by MCM-41-Supported Nickel Phosphides. Energy and Fuels 2007, 21 (2), 554.
(18) Ho, T. C. A Theory of Ultradeep Hydrodesulfurization of Diesel in Stacked-Bed Reactors. AlChE J. 2017, 0 (0), 1.
(19) Ho, T. C. Hydrodenitrogenation Property-Reactivity Correlation. Appl. Catal. A Gen. 2010, 378 (1), 52.
(20) Rodgers, R. P.; McKenna, A. M. Petroleum Analysis. Anal. Chem. 2011, 83 (12), 4665.
(21) Zeuthen, P.; Knudsen, K. G.; Whitehurst, D. D. Organic Nitrogen Compounds in Gas Oil Blends, Their Hydrotreated Products and the Importance to Hydrotreatment. Catal. Today 2001, 65 (2-4), 307.
(22) Wang, F. C. Y.; Qian, K.; Green, L. A. GC×MS of Diesel: A Two-Dimensional Separation Approach. Anal. Chem. 2005, 77 (9), 2777.
(23) Santos, J. M.; Galaverna, R. de S.; Pudenzi, M. A.; Schmidt, E. M.; Sanders, N. L.; Kurulugama, R. T.; Mordehai, A.; Stafford, G. C.; Wisniewski, A.; Eberlin, M. N. Petroleomics by Ion Mobility Mass Spectrometry: Resolution and Characterization of Contaminants and Additives in Crude Oils and Petrofuels. Anal. Methods 2015, 7 (11), 4450.
(24) Rodgers, R. P.; Klein, G. C.; Nowlan, V.; Marshall, A. G. Petroleomics: ESI FT-ICR MS Identification of Hydrotreatment-Resistant Neutral and Acidic Nitrogen Species in Crude Oil. ACS Div. Fuel Chem. Prepr. 2003, 48 (2), 574.
(25) Fu, J.; Kim, S.; Rodgers, R. P.; Hendrickson, C. L.; Marshall, A. G. Nonpolar Compositional Analysis of Vacuum Gas Oil Distillation Fractions by Electron Ionization Fourier Transform Ion Cyclotron Resonance Mass Spectrometry. Energy & Fuels 2006, 20 (2), 661.
(26) Tanaka, R.; Sato, S.; Takanohashi, T.; Hunt, J. E.; Winans, R. E. Analysis of the Molecular Weight Distribution of Petroleum Asphaltenes Using Laser Desorption-Mass Spectrometry. Energy and Fuels 2004, 18 (5), 1405.
(27) Rodgers, R. P.; Marshall, A. G. Petroleomics: Advanced Characterization of Petroleum-Derived Materials by Fourier Transform Ion Cyclotron Resonance Mass Spectrometry (FT-ICR MS). In Asphaltenes, Heavy Oils and Petroleomics; 2007; pp 63-93.
(28) Stanford, L. A.; Kim, S.; Rodgers, R. P.; Marshall, A. G. Characterization of Compositional Changes in Vacuum Gas Oil Distillation Cuts by Electrospray Ionization FT-ICR Mass Spectrometry. Energy & Fuels 2006, 20 (7), 1664.
(29) Oro, N. E.; Lucy, C. A. Analysis of the Nitrogen Content of Distillate Cut Gas Oils and Treated Heavy Gas Oils Using Normal Phase HPLC, Fraction Collection and Petroleomic FT-ICR MS Data. Energy and Fuels 2013, 27 (1), 35.
(30) Kanu, A. B.; Dwivedi, P.; Tam, M.; Matz, L.; Hill, H. H. Ion Mobility-Mass Spectrometry. J. Mass Spectrom. 2008, 43 (7), 1.
(31) Hill, H. H.; Siems, W. F.; Louis, R. H. St.; McMinn, D. G. Ion Mobility Spectrometry. Anal. Chem. 1990, 62 (23), 1201A.
(32) Ahmed, A.; Cho, Y.; Giles, K.; Riches, E.; Lee, J. W.; Kim, H. I.; Choi, C. H.; Kim, S. Elucidating Molecular Structures of Nonalkylated and Short-Chain Alkyl (N<5, (CH2)n) Aromatic Compounds in Crude Oils by a Combination of Ion Mobility and Ultrahigh-Resolution Mass Spectrometries and Theoretical Collisional Cross-Section Calculations. Anal. Chem. 2014, 86 (7), 3300.
(33) Lalli, P. M.; Klitzke, F.; Corilo, Y. E.; Pudenzi, M. A.; Pereira, R. C. L.; Bastos, W.; Daroda, R. J.; Eberlin, M. N. Petroleomics by Traveling Wave Ion Mobility—Mass Spectrometry Using CO 2 as a Drift Gas. Energy & Fuels 2013, 27, 7277.
(34) Fernandez-Lima, F. A.; Becker, C.; McKenna, A. M.; Rodgers, R. P.; Marshall, A. G.; Russell, D. H. Petroleum Crude Oil Characterization by IMS-MS and FTICR MS. Anal. Chem. 2009, 81 (24), 9941.
(35) Lalli, P. M.; Corilo, Y. E.; Rowland, S. M.; Marshall, A. G.; Rodgers, R. P. Isomeric Separation and Structural Characterization of Acids in Petroleum by Ion Mobility Mass Spectrometry. Energy & Fuels 2015, 29 (6), 3626.
(36) Ponthus, J.; Riches, E. Evaluating the Multiple Benefits Offered by Ion Mobility-Mass Spectrometry in Oil and Petroleum Analysis. Int. J. Ion Mobil. Spectrom. 2013, 16, 95.
(37) Ahmed, A.; Cho, Y. J.; No, M.-H.; Koh, J.; Tomczyk, N.; Giles, K.; Yoo, J. S.; Kim, S. Application of the Mason-Schamp Equation and Ion Mobility Mass Spectrometry to Identify Structurally Related Compounds in Crude Oil. Anal. Chem. 2011, 83 (1), 77.
(38) Panda, S. K.; Andersson, J. T.; Schrader, W. Characterization of Supercomplex Crude Oil Mixtures: What Is Really in There? Angew. Chem. Int. Ed. 2009, 48 (10), 1788.
(39) Eckert, P. A.; Roach, P. J.; Laskin, A.; Laskin, J. Chemical Characterization of Crude Petroleum Using Nanospray Desorption Electrospray Ionization Coupled with High-Resolution Mass Spectrometry. Anal. Chem. 2012, 84 (3), 1517.
(40) Kauppila, T. J.; Kuuranne, T.; Meurer, E. C.; Eberlin, M. N.; Kotiaho, T.; Kostiainen, R. Atmospheric Pressure Photoionization Mass Spectrometry. Ionization Mechanism and the Effect of Solvent on the Ionization of Naphthalenes. Anal. Chem. 2002, 74 (21), 5470.
(41) Pereira, R. C. L.; Simas, R. C.; Corilo, Y. E.; Vaz, B. G.; Klitzke, C. F.; Schmidt, E. M.; Pudenzi, M. A.; Silva, R. M. C. F.; Moraes, E. T.; Bastos, W. L.; et al. Precision in Petroleomics via Ultrahigh Resolution Electrospray Ionization Fourier Transform Ion Cyclotron Resonance Mass Spectrometry. Energy & Fuels 2013, 27 (12), 7208.
(42) Klitzke, C. F.; Corilo, Y. E.; Siek, K.; Binkley, J.; Patrick, J.; Eberlin, M. N. Petroleomics by Ultrahigh-Resolution Time-of-Flight Mass Spectrometry. Energy and Fuels 2012, 26 (9), 5787.
(43) Qian, K.; Rodgers, R. P.; Hendrickson, C. L.; Emmett, M. R.; Marshall, A. G. Reading Chemical Fine Print: Resolution and Identification of 3000 Nitrogen-Containing Aromatic Compounds from a Single Electrospray Ionization Fourier Transform Ion Cyclotron Resonance Mass Spectrum of Heavy Petroleum Crude Oil. Energy and Fuels 2001, 15 (2), 492.
(44) Jokuty, P. L.; Gray, M. R. Resistant Nitrogen Compounds in Hydrotreated Gas Oil from Athabasca Bitumen. Energy and Fuels 1991, 5 (6), 791.
(45) Marshall, A. G.; Rodgers, R. P. Petroleomics: Chemistry of the Underworld. Proc. Natl. Acad. Sci. 2008, 105 (47), 18090.
(46) Corilo, Y. E. PetroOrg Software; Florida State University, Omics LLC: Tallahassee, F L, 2014, http://www.petroorg.com.
(47) Ho, T. C.; Katritzky, A. R.; Cato, S. J. Effect of Nitrogen Compounds on Cracking Catalysts. Ind. Eng. Chem. Res. 1992, 31 (7), 1589.
(48) Korre, S. C.; Neurock, M.; Klein, M. T.; Quann, R. J. Hydrogenation of Polynuclear Aromatic Hydrocarbons. 2. Quantitative Structure/reactivity Correlations. Chem. Eng. Sci. 1994, 49 (24 PART A), 4191.
Anal. Chem. 1998, 70, 4929-4938 (“Electrospray Ionization High-Resolution Ion Mobility Spectrometry-Mass Spectrometry”).
Energy Fuels 2010, 24, 6321-6326 (“Tracking Neutral Nitrogen Compounds . . . Using Negative-Ion ESI FT-ICR MS”).
Anal. Chem., 2013, 85 (11), pp 5530-5534 (“Identification of Ion Series Using Ion Mobility Mass Spectrometry: The Example of Alkyl-Benzothiophene and Alkyl-Dibenzothiophene Ions in Diesel Fuels”).
U.S. Pat. No. 9,187,702 (Zahn et al.), issued Nov. 17, 2015.
U.S. Pat. No. 8,884,091 (Zahn et al.), issued Nov. 11, 2014.
U.S. Pat. No. 8,163,169 (Maesen, et. al.), issued Apr. 24, 2012.
US 2009/0298677 A1 (Radlowski et. al.), issued
U.S. Pat. No. 9,177,774 (Brunelli et al.), issued Nov. 3, 2015.
U.S. Pat. No. 7,723,115 (Qian et al.), issued May 25, 1010.
WO 02/48698 A1 (Roussis et al.), published Jun. 20, 2002.

The foregoing description of the invention, including any specific embodiment(s) of the invention and incorporated publication information, is primarily for illustrative purposes, it being recognized that variations might be used which would still incorporate the essence of the invention. Reference should be made to the following claims in determining the scope of the invention.

What is claimed is:

1. An ion mobility mass spectrometry (IMMS) method for determining the nitrogen compound speciation in a petroleum composition, comprising:
providing a sample of a petroleum composition;
combining the petroleum sample with a solvent and an ionization enhancer to form an IMMS sample;
providing the IMMS sample to an ion mobility mass spectrometer; and
obtaining the mass and drift time spectra of ionized IMMS sample components.

2. The method of claim 1, wherein the petroleum composition is selected from vacuum gas oil, vacuum resid, aromatic resid, unconverted crude oil, coker gas oil, cycle oil, straight run diesel, or a mixture thereof.

3. The method of claim 2, wherein the petroleum composition comprises vacuum gas oil.

4. The method of claim 1, wherein the solvent is selected from acetonitrile, dichloromethane, dichloroethane, tetrahydrofuran, methanol, ethanol, propanol, nitromethane, toluene, water, dimethylformamide, dimethylsulphoxide, or a mixture thereof.

5. The method of claim 4, wherein the solvent comprises dichloromethane.

6. The method of claim 1, wherein the ionization enhancer is selected from an organic acid, a halogenated organic acid, a carboxylic acid, a halogenated carboxylic acid, or a mixture thereof.

7. The method of claim 6, wherein the ionization enhancer comprises a halogenated organic acid.

8. The method of claim 7, wherein the ionization enhancer is fluoroacetic acid, chloroacetic acid, difluoroacetic acid, dichloroacetic acid, trifluoroacetic acid, trichloroacetic acid, or a mixture thereof.

9. The method of claim 8, wherein the ionization enhancer comprises trifluoroacetic acid.

10. The method of claim 1, wherein the mass spectrometer comprises an electrospray source, the IMMS sample is provided to the electrospray source, the IMMS sample is ionized by the electrospray source and ions from the IMMS sample are provided to the mass spectrometer.

11. The method of claim 1, wherein the ion mobility mass spectrometer comprises an ionization source, a travelling wave ion guide, a quadrupole, a tri-wave ion mobility separator, and a time-of-flight mass analyzer.

12. A method for determining the effectiveness of a hydroprocessing catalyst in removing nitrogen-containing compounds from a petroleum composition, comprising
providing a first petroleum sample from a petroleum composition that has not been hydroprocessed by contacting the first petroleum sample with a hydroprocessing catalyst;
providing a second petroleum sample from the petroleum composition that has been hydroprocessed by contacting the second petroleum sample with the hydroprocessing catalyst under effective hydroprocessing conditions;
separately combining each of the first and the second petroleum samples with a solvent and an ionization enhancer to form corresponding first and second IMMS samples;
separately providing the first and second IMMS samples to an ion mobility mass spectrometer;
obtaining the mass and drift time spectra of ionized IMMS sample components for each of the first and second IMMS samples; and
analyzing the spectra results to assign chemical species to selected peaks of the mass spectra and to determine the double bond equivalent (DBE) of the first and second IMMS samples relative to carbon number.

13. The method of claim 12, wherein the petroleum composition is selected from vacuum gas oil, vacuum resid, aromatic resid, unconverted crude oil, coker gas oil, cycle oil, straight run diesel, or a mixture thereof.

14. The method of claim 12, wherein the petroleum composition comprises vacuum gas oil.

15. The method of claim 12, wherein the solvent is selected from acetonitrile, dichloromethane, dichloroethane, tetrahydrofuran, methanol, ethanol, propanol, nitromethane, toluene, water, dimethylformamide, dimethylsulphoxide, or a mixture thereof.

16. The method of claim 12, wherein the solvent comprises dichloromethane.

17. The method of claim 12, wherein the ionization enhancer is selected from an organic acid, a halogenated organic acid, a carboxylic acid, a halogenated carboxylic acid, or a mixture thereof.

18. The method of claim 12, wherein the ionization enhancer comprises trifluoroacetic acid.

19. The method of claim 12, wherein the mass spectrometer comprises an electrospray source, the IMMS sample is provided to the electrospray source, the IMMS sample is ionized by the electrospray source and ions from the IMMS sample are provided to the mass spectrometer.

20. The method of claim 12, wherein the ion mobility mass spectrometer comprises an ionization source, a travelling wave ion guide, a quadrupole, a tri-wave ion mobility separator, and a time-of-flight mass analyzer.
